(19) United States
(12) Patent Application Publication
(10) Pub. No.: US 2003/0186908 A1
(43) Pub. Date: Oct. 2, 2003

(54) Microorganisms transformed with Ig-Like polypeptide and uses thereof

(75) Inventor: Martin Goldway, Timrat (IL)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Gavish-Galilee Bio Applications Ltd., Kibbutz Glil Yam (IL)

(21) Appl. No.: 10/277,471

(22) Filed: Oct. 22, 2002

(30) Foreign Application Priority Data
Oct. 22, 2001 (IL) 146108

(51) Int. Cl. A61K 48/00

(52) U.S. Cl. 514/44; 424/93.2

(57) ABSTRACT

The present invention relates to a composition for conferring to a subject, immunity against a gastrointestinal (GI) pathogen, particularly Salmonella. The composition of the invention comprises as an active ingredient at least one microorganism transformed with at least one vector comprising a nucleic acid sequence coding for an immunoglobulin-like polypeptide having specific binding affinity for said pathogen. The invention further relates to the single-chain variable fragment (ScFv) antibody molecule having affinity for Salmonella species, used by the invention and to methods for the treatment of infections of gastrointestinal enteropathogenic bacteria in a subject.

### FIELD OF THE INVENTION

The present invention relates to a vaccine composition and to methods of use thereof for treatment and/or prevention of mucosal pathogen infections in a subject in need. More particularly, the present invention relates to vaccine compositions and methods for inhibiting growth of bacterial pathogens in the mucosal surfaces, preferably gastrointestinal mucosal surfaces, of an animal.

### BACKGROUND OF THE INVENTION

The intestinal tract of all organisms is unique in its rich microbial milieu. For example, in humans it is estimated that the large bowel contains as many as 10¹³ bacteria/ml [Toskes, P. P., et al., In.: Gastrointestinal and Liver Diseases, 6 ed. W. B. Sauders Company, Philadelphia, P. 15231 (1998)]. The interaction between the host and resident micro-organisms is complex, The indigenous intestinal flora is vital to the host, and bacteria have a role in the maintenance of steady state homeostasis in mucosal surfaces [Toskes, P. P., et al., (1998) ibid.]. On the other hand, changes in the intestinal flora as a result of antibiotic treatment may result in bacterial growth that can potentially cause mucosal diseases by alteration of the normal intestinal flora and outgrowth of mucosal pathogens [Barlett, J. G., et al., In: Gastrointestinal and Liver Diseases. 6 ed. Mark Feldman, Bruce F. Scharschmidt and Marvin H. Sleisenger, eds. W. B. Saunders Company, Philadelphia, p. 1633(2) (1998)]. Moreover, antibiotic treatment may result in the emergence of antibiotic resistant strains of other, bystander-bacteria that reside in the intestinal tract. This problem is not unique to humans, since antibiotic treatment of livestock intended for human consumption, may result in the transfer of antibiotic-resistant bacteria to humans [Thwaites, R. T., et al., J. Clin. Pathol. 52:812 (1999); Helm, J. D., et al., Avian. Dis. 43:788 (1999); Keyes, K., et al., Antimicrob. Agents Chemother, 44(2):421-4 (2000); Aarestrup, F. M., et al., Vet. Rec. 146(3):76-8 (2000); Arcangioli, M. A., et al., J. Med. Microbiol. 49(1):103-10 (2000); Bradford, P. A., et al., J. Antimicrob. Chemother. 44:607 (1999); Farrington, L. A., et al., Adv. Exp. Med. Biol. 473:291 (1999)].

The Salmonella species are amongst the leading causes of food poisonings in humans [Mead, P. S., et al., Emerg. Infect. Dis. 5:607 (1999)]. Poultry products are the major reservoir of Salmonella [Hogue, A., et al., Rev. Sci. Tech. 16:542 (1997)]. Most infections result from the release and spread of intestinal contents of healthy chickens that are carriers of Salmonella. Moreover, some chicken carry the Salmonella in their ovaries and therefore the eggs are infected there within. Infection via eggs could also be prevented by avoiding colonisation of the GI tract. A number of measures have been tested in an attempt to reduce the load of zoonotic agents in animals and animal products in order to prevent the outbreak of food borne infections and intoxication in general, and of Salmonella specifically. For example, prophylactic administration of undefined mixtures or fecal bacteria obtained from adult chickens in to chicks was found to markedly reduce the colonisation of the gastrointestinal tract by Salmonella [Impey, C. S., et al., J. Appl. Bacteriol. 63:139 (1987)].

However, the widespread use of such undefined mixtures of organisms has been limited, since there is concern that human or animal pathogens maybe transferred as well [Stavric, S., Int. J. Food Microbiol. 15:245 (1992)].

The administration of single defined agents, isolated from the gastrointestinal tract, has neither the protective potential, nor the stability of undefined cultures [Stavric, S. (1992) ibid., Soerjadi, A. S., et al.. Aust. Vet. J. 54:549 (1978); Barrow, P. A., et al., J. Hyg. (Lond.) 96:161(1986); Barnes, E. M., et al., Br. Poult. Sci. 13:311 (1972)].

Recent advances in immunology, molecular biology, and peptide biochemistry have allowed for the construction of vaccines based on oral administration of antigenic subunits derived from recombinant viral or bacterial strains by the aid of defined bacterial vectors. The bacterial delivery systems which have been developed as vaccine vectors include Bacille Calmette-Guerin (BCG), Listeria monocytogenes, Salmonellae, Shigellae and Escherichia coli (E. coli) [Shata, M. T., et al., Mol. Med. Today 6(2):66-71 (2000)]. However, this approach is not completely satisfactory for protection from Salmonella infections, since an efficient Salmonella immunogenic subunit has not been identified [Levine, M. M., et al., P.N.G. Med. J 38:325(18) (1905)].

The present invention provides a new platform approach for the prevention of enteropathogenic infections, particularly, gastrointestinal infections, by utilising bacterial cells secreting an active immunoglobulin-like polypeptide having affinity to said enteropathogen.

A similar approach has been reported by Beninaty et al. [Beninaty C, et al., Nature Biotechnology 18:1060-1064 (2000)]. In this study, recombinant strains of the Gram positive Streptococcus gordonil, secreting or displaying a microbicidal single-chain antibody and stably colonizing rat vagina, were used in the treatment of experimental vaginitis caused by Candida albicans.

Although both the intestinal and female genital tissues are mucosal surfaces, and thus share some common features, they differ markedly. These differences are related both to the luminal contents of both tissues as well as to specific aspects of the immune system, For example, whereas the peristalsis and rapid movement of luminal contents is a central feature of the intestine, no such flow is present in the female vagina. This feature would have a cardinal effect on the current invention. Selection of an inappropriate organism for expression of the recombinant ScFv could be a direct cause for failure of this novel mode of immunization. Furthermore, the environmental conditions within these two mucosal surfaces differ as well. These include different and much more diverse flora residing in the intestine as compared to the vagina, and a pH gradient in the intestine, as compared to the female genital mucus surface. Such differences would have a major impact on the ability of the recombinant organism to colonize the mucosal surface. Therefore, the successful secretion of the ScFv within the intestine was remarkable.

Other important differences are directly linked to the immune system. Tius, it appears that whereas intestinal immune cells are educated within intestinal Peyer's patches, no such structure exists within the reproductive tract, and genital Ig secreting cells may in fact originate from the intestinal mucosa [Kutteh W. H. et al., Obstet. Gynecl, 71:56-60 (1988)]. This fact may hamper the production of immune response against localized pathogens such as Candida species and therefore, the exogenous application of the ScFv may have been of particular importance. In contrast, the contribution of the present invention in immunization for protection from Salmonella infection in the setup of the intestine itself is surprising and indicates that the selected vector and means of expression were properly designed so that their effect was apparent even within this fully functional milieu. Furthermore, the contents of the vaginal Ig secretion are regulated by the menstrual cycle [Schumacher G. F. B. in “Immunological aspects of infertility and fertility regulation” pp 93-135 Elsevier North Holland, New York (1980)]. In addition, within the vagina, IgG levels are greater than IgA levels and this phenomenon is probably controlled by the cervix [Jilanti R Int. Arch. Allergy Appl. Immunol 53:402-408 (1977)]. This fact markedly differs from the intestine in which IgA is the dominant isotype in the mucosal secretion. Since these Ig isotypes differ in important effector immune mechanisms such as complement fixation, these two mucosal surfaces cannot be expected to exert similar immune responses to pathogens. This difference is reiterated by the fact that candida species are pathogenic in the reproductive tract, whereas no significant pathology is associated with candida species in the intestine of the non-immunocompromised host. Taken together, significant differences exist between the female genital tract mucosa and the gastrointestinal tract.

Therefore, it is an object of present invention to provide compositions and methods for prevention of gastric luminal pathogens, and specifically, Salmonella, colonization and protection from its invasion into the intestinal mucosa and internal organs. The system described by the present invention composed of E. coli, which secrets an anti-Salmonella, single chain fragment variable (ScFv) antibody. As shown by the Examples, the protective effect is apparent both in vitro and in vivo. The present invention therefore provides a wide potential for treating other luminal pathogens, taking advantage of different bacterial vectors, suited for use in various anatomic compartments in an array of species.

### SUMMARY OF THE INVENTION

The first aspect of the present invention relates to a composition for conferring to a subject immunity against a mucosal pathogen. This composition comprises as an active ingredient at least one microorganism transformed with at least one vector. The vector comprises a nucleic acid sequence coding for at least one immunoglobulin-like polypeptide, having specific binding affinity for the pathogen. The composition of the invention optionally further comprises pharmaceutically and veterinarily acceptable carriers, adjuvants and/or diluents.

In a preferred embodiment of this aspect of the invention, the pathogen is a human or animal mucosal pathogen. More particularly, the mucosal pathogen is a mucosal surface enteropathogenic bacterium, and most preferably a gastrointestinal enterobacterium.

In a further particular yet non-limiting embodiment, the gastrointestinal enterobacterium is Salmonella and most preferably Salmonella enteriditis serogroup D.

In the compositions of the invention the immunoglobulin-like polypeptide is preferably an antibody or any fragments thereof, and most preferably, ScFv. This single chain variable fragment antibody, has binding affinity to enteropathogens such as gastrointestinal pathogens, preferably, Salmonella enterobacteria, and consists essentially of: a first polypeptide comprising the binding portion of the light chain variable region of an antibody; a second polypeptide comprising the binding portion of the heavy chain variable region of an antibody; and polypeptide linker linking both polypeptides. A preferred polypeptide linker is a glycine/serine, although it is to be appreciated that different linkers may be used for linking the two polypeptides.

In a specifically preferred embodiment, where the gastrointestinal entrobacterium is Salmonella enteriditis serogroup D, the ScFv is encoded by the nucleic acid sequence substantially as denoted by SEQ ID NO:1, and consists essentially of: a first polypeptide comprising the binding portion of the light chain variable region of an antibody against Salmonella enteriditis serogroup D, said first polypeptide being encoded by the nucleic acid sequence substantially as denoted by SEQ ID NO:2, a second polypeptide comprising the binding portion of the heavy chain variable region of an antibody against Salmonella enteriditis serogroup D, said second polypeptide being encoded by the nucleic acid sequence substantially as denoted by SEQ D NO:3, and a glycine/serine polypeptide linker linking both peptides, said linker being encoded by the nucleic acid sequence substantially as denoted by SEQ ID NO:4.

The transformed microorganism may be prokaryotic or eukaryotic. More particularly, the microorganism may be bacteria and also yeast. In a specifically preferred embodiment the composition of the invention may comprise as a bacterial cell a gram negative bacteria such as E. coli.

The composition of the present invention is intended for conferring to a subject immunity against a pathogen.

In a preferred embodiment, the composition of the invention is intended for conferring immunity to, and vaccinating human or a domestic animal. In a particular embodiment, the composition of the invention is intended for conferring immunity to domestic birds, more specifically, such domestic birds may be any one of chicken, ducks, geese, quails, pheasants and turkeys. Preferably, the composition of the invention is intended for vaccinating and conferring immunity to a chicken.

In a second aspect the invention relates to an isolated DNA coding for an immunoglobulin-like polypeptide that has specific affinity against enteropathogens.

In a preferred embodiment the DNA of the invention codes for single-chain variable fragment antibody that has specific affinity for entropathogens. This single-chain variable fragment antibody consists essentially of a first polypeptide comprising the binding portion of the light chain variable region of an antibody, a second polypeptide comprising the binding portion of the heavy chain variable region of an antibody; and a glycine/serine polypeptide linker linking the two polypeptides.

In another embodiment of this aspect, the DNA of the invention codes for ScFv specific for a gastrointestinal pathogen. Most particularly, the gastrointestinal pathogen is Salmonella, preferably Salmonella enteriditis serotype D.

A yet another aspect relates to a replicable cloning or expression vehicle comprising the DNA molecule of the invention. In a preferred embodiment the vehicle of the invention may be a phagemid.

Another aspect relates to a host cell transformed with any one of the expression vehicles of the present invention. The host cell of the invention may be prokaryotic or eukaryotic, preferably bacterial or yeast cell, and most preferably E. coli.

The host cell of the invention is capable of in vivo producing a biologically active immunoglobulin-like peptide, preferably a single chain variable fragment antibody. This antibody recognizes and specifically binds to the gastrointestinal pathogen.

In another preferred embodiment, when introduced to a subject, the host cell of the invention is capable of in vivo producing single chain variable fragment antibodies that can block colonization of the enteropathogens in said subject and/or their invasion into the host. Preferably, the ScFv antibody of the invention may block the colonization of different enterobacteria in the mucosal surfaces of the animal, and most preferably in gastrointestinal tract.

The invention further relates to a single-chain variable fragment antibody having affinity for Salmonella. This single-chain variable fragment antibody consists essentially of a first polypeptide comprising the binding portion of the light chain variable region of an antibody, a second polypeptide comprising the binding portion of the heavy chain variable region of an antibody and a glycine/serine polypeptide linker linking the two polypeptides.

In a specifically preferred embodiment, the single-chain variable fragment antibody is encoded by the nucleic acid sequence substantially as denoted by SEQ ID NO:1.

Yet further, the invention relates to a method for treating infections of human or animal mucosal pathogen, preferably infections of enteropathogens in the intestinal tract of a subject. The method comprises the administration of microorganisms transformed with a DNA encoding at least one immunoglobulin-like peptide, preferably the single-chain antibody molecules of the invention, or expression vector comprising the same. The single-chain antibody comprises a first polypeptide comprising the binding portion of the light chain variable region of an antibody, a second polypeptide comprising the binding portion of the heavy chain variable region of an antibody; and a glycine/serine polypeptide linker linking the two polypeptides.

The method of the invention may alternatively employ a veterinary composition comprising the transformed microorganisms of the invention.

In a preferred embodiment the method of the invention is intended for the treatment of pathogenic infections by an enterobacterium, preferably by gastrointestinal enterobacterium. A particular example is Salmonella particularly Salmonella enteriditis serogroup D.

For the treatment of infection by this pathogen, the method of the invention employs microorganisms producing an immunoglobulin-like polypeptide, preferably a single chain variable fragment (ScFv) antibody which is encoded by the nucleic acid sequence substantially as denoted by SEQ ID NO:1, or compositions comprising the same. The single chain variable fragment (ScFv) antibody consists essentially of a first polypeptide comprising the binding portion of the light chain variable region of an antibody against Salmonella enteriditis serogroup D, encoded by the nucleic acid sequence substantially as denoted by SEQ ID NO:2, a second polypeptide comprising the binding portion of the heavy chain variable region of an antibody against Salmonella enteriditis serogroup D, encoded by the nucleic acid sequence substantially as denoted by SEQ ID NO:3, and a glycine/serine polypeptide linker linking said first and second polypeptides, said linker being encoded by the nucleic acid sequence substantially as denoted by SEQ ID NO:4.

A preferred embodiment relates to the method of the invention wherein upon administration to a subject, the microorganism is capable of in vivo producing a single chain variable fragment antibody in the subject's intestine. This antibody recognizes and specifically binds to gastrointestinal pathogens. Moreover, the single chain variable fragment antibody is capable of blocking colonization of gastrointestinal pathogens in the intestinal tract of the treated animal.

In a specifically preferred embodiment the subject may be any mammalian subject, preferably a human or may be any domestic animal. More specifically, the domestic animals may be domestic birds, particularly chicken, ducks, geese, quails, pheasants and turkeys. Most preferably, the bird is a chicken.

The composition may be administrated via drinking water, feed, spraying, oral gavage and/or directly into the digestive tract.

In yet another specifically preferred embodiment the method of the invention may further comprise the step of controlling the expression of the immunoglobulin-like polypeptide. More specifically, controlling of the expression of a single chain variable fragment antibody by continuously inducing its production, for example by supplementing drinking water or feed with IPTG (isopropyl β-D-thiogalactoside).

### BRIEF DESCRIPTION OF THE FIGURES

FIG. 1—Nucleotide and Amino Acids Sequence of the Anti-Salmonella Serogroup D ScFv

The figure shows the nucleotide (also denoted by SEQ ID NO: 1) and amino acids (also denoted by SEQ ID NO: 5) full sequences of the anti-Salmonella serogroup D ScFv: The nucleic acid sequence comprising the light chain, nucleotides 1-339 (also denoted by SEQ ID NO: 2); linker, nucleotides 340-381 (also denoted by SEQ ID NO: 4); heavy chain, nucleotides 882-732 (also denoted by SEQ ID NO: 3); E-tag, nucleotides 742-780; stop codon indicated by (*).

FIGS. 2A-2B—The Anti-Salmonella ScFv is Expressed by E. coli and Secreted into the Growth Medium

2A. Western blot of periplasmic extract. Bacteria were grown in liquid medium. Periplasmic contents were extracted as described and size separated using a 12% SDS acrylamide gel. MW—molecular weight marker. Lane 1-ScFv expressed by E. coli transformed with pAM260.

2B. Western blot of culture media. Lane 1, control, E. coli transformed with pCANTAB5E-c and grown in 2YT medium supplemented with sucrose; lane 2, control, E. coli transformed with pCANTAB5E-c grown in 2YT medium supplemented with glucose; lane 3, E. coli transformed with pAM260 grown in 2YT supplemented with sucrose; lane 4, E. coli transformed with pAM260 grown in 2YT supplemented with glucose; lane 5, control, RPAS-ScFv provided by Pharmacia. MW—molecular weight marker.

FIGS. 3A-3B—The ScFv Detects Unfractionated Salmonella

Periplasmic fluids were extracted from E. coli that was transformed with pAM260 and from control bacteria transformed with pCANTAB5E-c. The extracts were tested by ELISA for their ability to detect unfractionated Salmonella.

3A. Detection with goat anti-mouse IgG. 1, blank; 2, hybricloma medium containing whole anti-Salmonella antibody molecules.

3E. Detection with anti E-tag antibody. 1, blank; 2, periplasmic extract of E. coli that was transformed with control plasmid pCANTAB5E-c; 3, periplasmic extract of E. coli that was transformed with pAM260. One experiment is a representative of three. Error bars represent differences between individual ELISA wells.

FIG. 4—ScFv Prevents Invasion of Salmonella into HT-29 Intestinal Cells

HT-29 cells were grown to confluence. Salmonella were pre-treated with periplasmic extracts containing ScFv or controls and were added to the culture for 24 hours. Following incubation, cells were lysed and the Salmonella in the lysates were grown overnight, after which OD⁶⁰⁰ was determined. 1, blank; 2, cell culture medium; 3, hybridoma medium; 4, Salmonella; 5, Salmonella to which hybridoma 41.11 growth medium was added; 6, Salmonella to which periplasmic extract from E. coli transformed with pAM260 was added; 7, Salmonella to which E. coli transformed with control plasmid pCANTAB5E-c was added. Results are mean and SE of three different experiments. Error bars represent differences between individual experiments.

FIG. 5—ScFv Reduces IL-8 Secretion from HT-29 Cells Following Incubation with Salmonella

HT-29 cells were grown to confluence. Salmonella were pre-treated for 1 hr. with ScFv or controls and added to the culture for 24 hrs. Following incubation, supernatants were collected and assayed for IL-8 secretion. 1, blank; 2, growth medium; 3, Salmonella; 4, Salmonella to which hybridoma 41.11 medium was added; 5, control, Salmonella to which E. coli HB2151 carrying pCANTAB5E was added; 6, Salmonella to which E. coli HB2151 carrying pAM260 was added. One experiment is representative of three, error represents differences between ELISA wells. Mean separation in each treatment by Ducan's multiple range test, P=0.05.

FIG. 6—ScFv Expressed on Bacteria Surface Prevents Invasion of Salmonella into HT-29 Intestinal Cells

A culture of 4.5×10⁸ E. coli expressing the ScFv (HB 2151/pAM260) or, 4.5×10⁸, negative control E. coli containing the vector only (HB 2151), were induced by IPTC and were then co-cultured with 4×10⁵ rifampicin-sensitive Salmonella. Bacteria cultures were then added to confluent HT-29 cells. Supernatants from hybridoma 41.11 (Hyb) and periplasmic fluid from bacteria expressing the ScFv (periplasma) served as positive controls. Subsequently, the cells were washed and then lysed using hypotonic shock and intracellular Salmonella were inoculated for culture in brain heart liquid medium. Bacteria were quantitated by OD₆₀₀ measurements.

### DETAILED DESCRIPTION OF THE INVENTION

In the model system presented in the Examples section of the present application, specific anti-Salmonella ScFv was designed to be secreted by E. coli and was shown to significantly inhibit the invasion of Salmonella into epithelial cells in vitro. Moreover, administration of a composition comprising these antibody-producing E. coli host cells to newborn chickens significantly reduced in vivo the number of Salmonella cells in the intestinal lumen of the chickens and reduced mucosal invasion.

Therefore, a particular application of the present invention is the inhibition of gastrointestinal enteropathogenes, preferably, Salmonella growth in the intestines of any subject and preferably, domestic animals, particularly for preventing of infection of human consumers of food products originating from the animals.

Thus, the model system disclosed in present application provides a novel platform for immunizing any animal against variety of gastrointestinal enteropathogenic infections.

A first aspect of the present invention therefore relates to a composition for conferring to an animal immunity against a pathogen. This composition comprises as an active ingredient at least one microorganism transformed with at least one vector. The vector comprises nucleic acid sequence coding for at least one immunoglobulin-like polypeptide that has specific binding affinity to said pathogen. The composition of the invention optionally further comprises pharmaceutically and veterinarily or pharmaceutically acceptable carrier, adjuvant or diluent. It is to be appreciated that the microorganism according to the invention may be transformed by a number of vectors expressing a number of immunoglobulin-like polypeptides, preferably ScFv aimed at different antigenic targets.

As used to describe the present invention, a “pathogen” is a microorganism causing disease in a host. The pathogenic microorganism infects the host animal and the consequence of such infection is deterioration in the health of the host. Pathogenic microorganisms envisioned by the present invention include, but are not limited to, microorganisms such as bacteria, viruses, yeast, fungi and other protozoa.

In a preferred embodiment of this aspect, the mucosal pathogen is a human enteropathogenic bacterium. More particularly, this enteropathogenic bacterium is a mucosal surface enteropathogenic bacterium.

Thus, the composition of the invention may also be applied to different mucosal surfaces containing a natural flora. These include the respiratory system, the oral cavity and the conjunctiva. Proper selection of the microorganism that may serve as the transformed host and express the immunoglobulin-like polypeptides will allow for stable expression under the different conditions.

In a most preferred embodiment the mucosal surface enteropathogenic bacterium may be any gastrointestinal enterobacterium. Of particular interest are gastrointestinal enterobacteria such as Salmonella and most preferably Salmonella enteriditis serogroup D.

Salmonella spp. are causative agents of human intestinal disease. As many as two million cases of salmonellosis occur annually in the United States [Krienberg et al., Food Technology, pages 77, 80, 81 and 98 (1987)]. These pathogenic microorganisms may colonize the gastrointestinal tract of domestic animals, especially birds, without any deleterious effects on the birds, and although some colonized birds can be detected, asymptomatic carriers can freely spread the microorganisms during poultry production and processing, resulting in further contamination of both live birds and carcasses. Poultry makes the primary reservoir for Salmonella in the food supply.

It is to be appreciated that the immunoglobulin-like polypeptide of the invention may be an antibody or any fragments thereof. The term “antibody” is meant to include both intact molecules as well as fragments thereof, such as, for example, Fab and F(ab′)₂, which are capable of binding antigens. Fab and F(ab′)₂ fragments lack the Fc fragment of the intact antibody, clear more rapidly from the circulation, and may have less non-specific tissue binding than an intact antibody [Wahl et al., J. Nucl. Med. 24:316-325 (1983)].

According to a specific embodiment, the immunoglobubin-like polypeptide is preferably a ScFv antibody. This single chain variable fragment antibody has binding affinity to gastrointestinal enterobacteria, and consists essentially of:

(a). first polypeptide comprising the binding portion of the light chain variable region of an antibody;

(b). a second polypeptide comprising the binding portion of the heavy chain variable region of an antibody; and

(c). a polypeptide linker linking said first and second polypeptides (a) and (b).

The terms “single chain variable fragment antibody”, “single chain antibody” or “ScFv” are used interchangeably herein. They are genetically engineered molecules structurally defined as comprising the binding portion of a first polypeptide from the variable region of an antibody (light chain), associated with the binding portion of a second polypeptide from the variable region of an antibody (heavy chain), the two polypeptides being joined by a peptide linker linking the first and the second polypeptides into a single polypeptide chain. The single polypeptide chain thus comprises a pair of variable regions connected by a polypeptide linker. These regions may associate to form a functional antigen-binding or antigen recognition site.

Multivalent single chain antibodies can be also employed in the present invention. This term means two or more single chain antibody fragments covalently linked by a peptide linker. The antibody fragments can be joined to form bivalent, trivalent or multivalent antibodies having one or more antibody fragments joined by an additional inter-peptide linker.

The single chain antibody fragments for use in the present invention can also be derived from the light and/or heavy chain variable domains of any antibody. The light and the heavy chain variable domains may be specific for the same antigen or can be directed against different antigens.

As described in Example 1, the immunoglobulin-like polypeptide of the invention was prepared using RNA extracted from hybridoma clone 41.11 specific for Salmonella, but a large source of hybridomas and their corresponding monoclonal antibodies is available for the preparation of sequences coding for the heavy (H) and light (L) chains of the variable region of different monoclonal antibodies against Salmonella or other pathogens. Furthermore, such hybridomas may be produced according to the therapeutic target.

Further, multiple epitopes of the pathogen could be defined by the use of phage display libraries and efficient immunization may be achieved by the administration of a bacterial vector that produces a “cocktail” of protecting antibodies.

The variable regions of both heavy and light chains show considerable variability in structure and amino acid composition from one antibody molecule to another, whereas the constant regions show little variability. The term “variable” as used herein refers to the diverse nature of the amino acid sequences of the antibody heavy and light chain variable regions. Each antibody recognizes and binds an antigen through the binding site defined by the association of the heavy and light chain variable region into a Fv area. The light-chain variable region VL and the heavy-chain variable region VH of a particular antibody molecule have specific amino acid sequences that allow the antigen-binding site to assume a conformation that binds to the antigen epitope recognized by that antibody.

The peptide linker joining the VH and VL domains to form a ScFv and the peptide linker joining two or more ScFv to form a multivalent single chain antibody may substantially have the same amino acid sequence.

“Linker” as used herein is a peptide, usually between two and 150 amino acid residues in length, that serves to join two protein domains in a multi-domain fusion protein. Examples of specific linkers can be found, for instance, in Hennecke et al. [Protein Eng. 11:405-410, (1998)] and U.S. Pat. Nos. 5,767,260 and 5,856,456.

Depending on the domains being joined, and their eventual function in the fusion protein, linkers may be from about 10 to about 100 amino acids in length or preferably from about 35 to about 50 amino acids in length, though these limits are given as general guidance only. The tendency of the fusion proteins to form specific and non-specific multimeric aggregations is influenced by linker length [Alfthan et al., Protein Eng. 8:725-731, (1998)]. Thus, shorter linkers will tend to promote multimerization, while longer linkers tend to favour maintenance of monomeric fusion proteins. Aggregation can also be minimized through the use of specific linker sequences, as demonstrated in U.S. Pat. No. 5,856,456.

Linkers may be chosen to have more or less secondary character (e.g. helical character, U.S. Pat. No. 5,637,481) depending on the conformation desired in the final fusion protein. The more secondary character a linker possesses, the more constrained the structure of the final fusion protein will be. Therefore, substantially flexible linkers that are substantially lacking in secondary structure allow flexion of the chimeric protein at the linker.

A linker is capable of retaining a binding domain of a protein in binding proximity of a target site when the linker is of a sufficient length and flexibility to allow specific interaction between both binding domains and the target corresponding sites.

In a specifically preferred embodiment, where said gastrointestinal entrobacterium is Salmonella enteriditis serogroup D, the single chain variable fragment antibody (ScFv) is encoded by the nucleic acid sequence substantially as denoted by SEQ ID NO:1, and consists essentially of:

(a). a first polypeptide comprising the binding portion of the light chain variable region of an antibody against Salmonella enteriditis serogroup D, said first polypeptide being encoded by the nucleic acid sequence substantially as denoted by SEQ ID NO:2;

(b). a second polypeptide comprising the binding portion of the heavy chain variable region of an antibody against Salmonella enteriditis serogroup D, said second polypeptide being encoded by the nucleic acid sequence substantially as denoted by SEQ ID NO.3; and

(c). a glycine/serine polypeptide linker linking said first and second polypeptides, said linker being encoded by the nucleic acid sequence substantially as denoted by SEQ ID NO:4.

The transformed microorganism of the invention may be prokaryotic and eukaryotic. More particularly, the transformed microorganism may be bacterial and yeast. In a specifically preferred embodiment the composition of the invention may comprise as transformed bacterial cells a gram negative bacteria such as E. coli.

The composition of the present invention is intended for conferring to a subject immunity against a pathogen, preferably, a gastrointestinal pathogen.

According to the invention, the subject may be any mammal, preferably a human. Alternatively, said subject may be any domestic animal.

In a preferred embodiment the composition of the invention is applicable to any domestic animal and particularly to birds that are raised for human consumption, which could serve as carriers of the target pathogens. More specifically, the birds may be chicken, ducks, geese, quails, pheasants and turkeys. Preferably, the birds are chicken.

In a second aspect the invention relates to an isolated DNA molecule coding for an immunoglobulin-like polypeptide having specific binding affinity for a mucosal surface enteropathogen. This pathogen may be a human enteropathogenic bacterium, preferably a gastrointestinal enterobacterium.

In a specific embodiment of the present aspect, the DNA of the invention codes for a single-chain variable fragment antibody (ScFv), as an immunoglobulin-like polypeptide. The single-chain variable fragment antibody that has specific affinity for gastrointestinal pathogens. This single-chain variable fragment antibody consists essentially of:

(a). a first polypeptide comprising the binding portion of the light chain variable region of an antibody;

(b). a second polypeptide comprising the binding portion of the heavy chain variable region of an antibody; and

(c). a polypeptide linker linking said first and second polypeptides (a) and (b).

In a particular example, the gastrointestinal pathogen may be Salmonella species, preferably, Salmonella enteriditis serotype D.

In this particular example, the DNA of the invention is encoded by the nucleic acid sequence substantially as denoted by SEQ ID NO-1.

The invention further relates to a replicable cloning or expression vehicle comprising the DNA molecule of the invention. In a preferred embodiment the vehicle of the invention may be a phagemid, preferably, the pAM20 phagemid. A specific example is the expression vehicle designated pAM260, as described in the Examples.

Expression vehicles for production of the molecules of the invention include plasmids, phagemids or other vectors. “Vectors”, as used herein, encompass plasmids, viruses, bacteriophage, integratable DNA fragments, and other vehicles which enable the integration of DNA fragments into the genome of the host. Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or an eukaryotic promoter expression control system. Such system typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

A vector may additionally include appropriate restriction sites, antibiotic resistance or other markers for selection of vector containing cells. Plasmids are the most commonly used vectors but other forms of vectors which serve an equivalent function and which are, or become, known in the art are suitable for use herein. See, e.g., Pouwels et al. Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass. (1988), which are incorporated herein by reference.

In general, such vectors contain, in addition specific genes, which are capable of providing phenotypic selection in transformed cells. The use of prokaryotic and eukaryotic viral expression vectors to express the genes coding for the polypeptides of the present invention is also contemplated.

As disclosed herein, recombinant phagemids have been prepared which, when used to transform bacterial host cells, permit the secretion of foreign protein outside the cytoplasmic membrane of the host cell.

The vector is introduced into a host cell by methods known to those of skilled in the art. Introduction of the vector into the host cell can be accomplished by any method that introduces the construct into the cell, including, for example, calcium phosphate precipitation, microinjection, electroporation or transformation, See, e.g., Current Protocols in Molecular Biology, Ausuble, F. M., ed., John Wiley & Sons, N.Y. (1989).

Another aspect relates to host cell transformed with any one of the expression vehicles of the present invention. Suitable host cells include prokaryotes, lower eukaryotes, and higher eukaryotes. Prokaryotes include gram negative and gram positive organisms, e.g., E. coli and B. subtilis. Lower eukaryotes include yeast, S. cerevisiae and Pichia, and species of the genus Dictyostelium.

“Host cell” as used herein refers to cells which can be recombinantly transformed with DNA encoding at least one immunoglobulin-like polypeptide according to the invention, or with expression vectors comprising the same, that were constructed using recombinant DNA techniques. A drug resistance or other selectable marker is intended in part to facilitate the selection of the transformants. Additionally, the presence of a selectable marker, such as drug resistance marker may be of use in keeping contaminating microorganisms from multiplying in the culture medium. Such a pure culture of the transformed host cell would be obtained by culturing the cells under conditions which require the induced phenotype for survival.

In a preferred embodiment the host cell of the invention may be a bacterial or yeast cell and most preferably, a gram negative bacterium such as E. coli.

One preferred host is a yeast cell. Yeast provide substantial advantages for the production of immunoglobulin light and heavy chains. Yeast carry out post-translational peptide modifications. Yeast gene expression systems can be routinely evaluated for the levels of heavy and light chain production, protein stability and secretion.

Among bacterial hosts which may be utilized as transformation hosts, E. coli HB2151 cells [k12Δ(lac-pro), ara, nalr, thilF [proAB, lacq, lacZΔM15), (Pharmacia Biotech Inc New Jersey U.S.A) is particularly useful. Other microbial strains which may be used include other entrobacteria such as Serratia marcescens.

The production of antibodies and antibody fragments in bacterial systems has been pursued by workers in the field, particularly in E. coli expression systems. There are several advantages to E. coli expression systems, including well-studied and convenient gene technology, which permits constructs to be made easily and directly expressed. Accordingly, the expression is followed by secretion to the periplasm of the recombinant cell. The expression of antibody genes in bacteria was reported by Cabilly et al., Proc. Natl. Acad. Sci. USA 81:3273-3277 (1984) and Boss et al., Nucleic Acid Res. 12:3791-3806 (1984). These reports show cytoplasmic expression and rather variable yield.

The host cell of the invention is capable of in vivo producing and preferably secreting, a biologically active immuinoglobulin-like polypeptide, preferably single chain variable fragment antibody. This antibody recognizes and specifically binds to mucosal surface enteropathogen, preferably a gastrointestinal pathogen.

In another preferred embodiment when introduced to a subject, preferably a domestic animal, the host cell of the invention is capable of in vivo producing a single chain variable fragment antibody. This antibody blocks colonization of gastrointestinal pathogens in the intestinal tract of said subject.

The invention further relates to a fusion protein which is a single-chain variable fragment antibody molecule having affinity for Salmonella. This single-chain variable fragment antibody consists essentially of:

(a). a first polypeptide comprising the binding portion of the light chain variable region of an antibody;

(b). a second polypeptide comprising the binding portion of the heavy chain variable region of an antibody; and

(c). a polypeptide linker linking said first and second polypeptides (a) and (b).

In a specifically preferred embodiment the single-chain variable fragment antibody molecule is encoded by the nucleic acid sequence substantially as denoted by SEQ ID NO:1.

A heterologous fusion protein is a fusion protein made of segments, which are naturally not normally fused in the same manner. Thus, the fusion product of variable light and variable heavy chains linked by the peptidic linker, is a continuous protein molecule having sequences fused by a typical peptide bond, typically made as a single translation product and exhibiting properties derived from each source peptide.

The immunoglobulin-ike polypeptide or preferably the ScFv antibody is said to be “having specific binding affinity” to a pathogen, if it is capable of specifically reacting with a molecule expressed on the surface of that pathogen. The term “epitope” is meant to refer to that portion of any molecule capable of being bound by a ScFv antibody that can also be recognized by that antibody. Epitopes or “antigenic determinants” usually consist of chemically active surface groupings of molecules such as amino acids or sugar side chains, and have specific three-dimensional structural characteristics as well as specific charge characteristics.

An “antigen” is a molecule or a portion of a molecule capable of being bound by an antibody or fragments thereof (e.g., ScFv), which is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies which may be evoked by other antigens.

In yet another aspect the invention relates to a method for treating infections of mucosal surface enteropathogens in a subject in need. The method of the invention comprises the administration to said subject of microorganisms transformed with a vector expressing at least one immunoglobulin-like polypeptide or of pharmaceutically or veterinary composition comprising the same. The polypeptide of the invention may preferably be a ScFv that has specific binding affinity to the pathogen.

According to a specific embodiment, the invention relates to a method of treating infections of human enteropathogenic bacteria in the intestinal tract of a subject. The method comprises the administration of an effective amount of microorganisms transformed with a vector expressing at least one single-chain antibody molecules. The single-chain antibody molecules comprise:

(a). a first polypeptide comprising the binding portion of the light chain variable region of an antibody;

(b). a second polypeptide comprising the binding portion of the heavy chain variable region of an antibody; and

(c). a glycine/serine polypeptide linker linking said first and second polypeptides (a) and (b).

The method of the invention may alternatively employ an effective amount of a veterinary or pharmaceutical composition comprising the microorganisms of the invention.

As used herein, “effective amount” means an amount necessary to achieve a selected result. For example, an effective amount of the composition of the invention useful for reducing the titer of an elected pathogenic microorganism in the gastrointestinal tract would be an amount that achieves the selected result of reducing the titer of the microorganism. Such amount will be determined by the veterinary attendant, and will depend on age, weight and disease of the treated subject or animal.

The novel approach that was explored in the present invention may be relevant in the treatment of a broad array of enteropathogens. Several aspects of this novel immunisation technique may be manipulated to allow for additional versatility in applying this approach. The secretion of the ScFv persists as long as the secreting bacteria survive in the colon. The rich microbial milieu in the colon may allow for the use of different bacteria that can serve as vectors for the expression of the ScFv. Such variability may enable to determine the preferred site for expression of the antibody or, to apply various stimuli to induce or block secretion. For example, E. coli was chosen as a vector for the secretion of the ScFv, since it is a prevalent bacterium in the gastrointestinal flora in chickens [Barnes, et al., Br. Poult. Sci. 13:311 (1972.); Leitner, et al., Avian. Dis. 36:211 (1992)] and its use was predicted to result in wide distribution of the ScFv-secreting bacteria. Other suitable hosts and vectors may be used.

In a preferred embodiment the method of the invention is intended to treat a subject such as any mammalian subject, preferably, human or alternatively, any domestic animal infections by enterobacteria, particularly, gastrointestinal enterobacteria.

A specifically preferred example for such pathlogen may be Salmonella and particularly, Salmonella enteriditis serogroup D. For treatment of infection by this pathogen, the method of the invention employs an effective amount of a composition or microorganism producing single chain variable fragment antibody (ScPv) which is encoded by the nucleic acid sequence substantially as denoted by SEQ ID NO:1. The single chain variable fragment antibody (ScFv) consists essentially of:

(a). a first polypeptide comprising the binding portion of the light chain variable region of an antibody against Salmonella enteriditis serogroup D, encoded by the nucleic acid sequence substantially as denoted by SEQ ID NO:2;

(b). a second polypeptide comprising the binding portion of the heavy chain variable region of an antibody against Salmonella enteriditis serogroup D, encoded by the nucleic acid sequence substantially as denoted by SEQ ID NO:3; and

(c), a glycine/serine polypeptide linker Linking said first and second polypeptides and encoded by the nucleic acid sequence substantially as denoted by SEQ ID NO:4.

In the method described herein resident intestinal bacteria were designed to continuously release an immunoglobulin-like polypeptide, preferably single chain variable fragment (ScFv) antibody as a novel measure of intestinal immunization. The method has the advantage of enabling in vivo release of a therapeutic agent (ScFv) that is aimed against a specific enteropathogen.

Furthermore, a preferred embodiment relates to the method of the invention wherein upon administration to an animal, the microorganism is capable of in vivo producing a single chain variable fragment antibody in the animal mucosal surface for example, intestine. This antibody recognizes and specifically binds to gastrointestinal pathogens. Moreover, the single chain variable fragment antibody can block colonization of enteropathogens in the intestinal tract of the treated animal.

The method of the invention also has the potential ability to elicit different types of protective mechanisms. Thus, using this method, immunity against different pathogens does not depend on classical recognition by the immune system. Important antigenic domains, that are not normally immunogenic, may be exposed in vitro. The immunity to these antigens may be enhanced by the use of appropriate haptens and adjuvants. The antibodies that would be produced and cloned in the relevant hybridomas could then be used for expression and immunisation.

The recognition of pathogens by luminal antibodies may be manipulated to produce an active immune response. For example, mounting an efficient intestinal immune response depends in part on active uptake of the relevant antigens by M cells that overlay the payers patches [Frey, et al., Behring. Inst. Mitt. 376:376-89 (1997)].

The sampling of such antigens may be facilitated by binding of antigen aggregates to M cells [Nurmi, et al., Nature 241:210(25) (1973)]. Such binding could result in the mounting of an active immune response. Thus, the determination of the nature of the antibody that is secreted from the bacteria, can lead to the development of active immunisation that will potentially defend the host against future infections and will obviate the need for continuous administration of the bacterial vector.

The method of the invention is intended for treating subjects like humans or domestic animals. In a specifically preferred embodiment the domestic animal may be a domestic bird selected from the group consisting of chicken, ducks, geese, quails, pheasants and turkeys. Most preferably, the birds are chicken.

In the method of the invention the composition may be administrated via drinking water, food, spraying, oral gavage and/or directly into the digestive tract.

The transformed hosts of the invention may be administered directly to the animal to be treated, or it may be desirable to administer to the animal compositions comprising the transformed hosts and it may be desirable to pharmaceutically or veterinarily add acceptable carriers, adjuvants or diluents to the composition prior to its administration. Therapeutic formulations may be administered in any conventional dosage formulation. Formulations typically comprise at least one active ingredient, as defined above, together with one or more acceptable carriers.

Each carrier should be pharmaceutically and veterinarily acceptable in the sense of being compatible with the other ingredients and not harmful to the treated subject. The formulations may conveniently be presented in unit dosage form and may be prepared by any methods well known in the art of pharmacy and veterinary.

The composition of the invention may be mixed with nutritive feed material or water supplies for the animal. It is contemplated however that the effective composition can either be mixed with the nutritive feed material or water or fed to the animal separately. The effective composition must be provided in an amount effective to inhibit the growth of any enteropathogenic bacteria such as Salmonella. This amount will vary depending upon the size of the treated animal. Birds will require smaller quantities of the effective composition than, for example, cattle, to inhibit intestinal Salmonella. Effective amount can readily be determined by the skilled veterinarian, and will depend on age, weight and disease of the animal.

The feed composition for the treated animals may contain the effective composition of the invention or the host cells transformed with the vector of the invention.

The use of the composition of the invention as a live vaccine should be controlled tightly. Changes in growth conditions can allow for the manipulation of antibody production by the bacteria. As described in Example 3, an inducing agent (IPTG) was added to the King water of the chicks together with the transformed bacteria to enable continued induction of ScFv synthesis in the colon. Such manipulation of the synthetic activity of the bacterial vectors may permit precise manipulation of the level of the immunogenic effect. Introduction of bacteria with established antibiotic sensitivity might allow for a defined safety profile.

Thus, in yet another specifically preferred embodiment the method of the invention may further comprise the step of controlling the expression of the single chain variable fragment antibody by continuously inducing or reducing its production. For example, such control by induction, may be achieved by supplementing drinking water with IPTG.

The novel approach presented herein may provide a new platform strategy to treat infections of mucosal surfaces and preferably, gastrointestinal tract. It has the advantage of allowing rapid onset of action and rapid oral delivery of immunization to large populations. Furthermore, this method may be employed in cases of immunodeficiency, or when the pathogen is not immunogenic. Importantly, it allows for the specific treatment of infectious disease without the use of antibiotics, thus avoiding their environmental deleterious effects.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise. Thus, for example, reference to composition containing “a microorganism” may include a mixture of two or more microorganisms.

The terms “bacteria” and “bacterium” are used herein interchangingly.

The various publications referred to herein, including publications cited therein, are fully incorporated herein by reference.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

Plasmids

The pCANTAB5E phagemid vector (Pharmacia Biotech Inc. New Jersey U.S.A.) was used for the expression of soluble antibody ScFv fragment. pCANTAB5E is designed such that the antibody variable region genes can be cloned between the leader sequence and the main sequence of the M13 gene 3 (gP3). The fusion protein that is expressed retains the function of both parent proteins. pCANTAB5E also contains a sequence encoding a peptide tag (“E-tag”) followed by an amber translational stop codon at the junction between the cloned ScFv and the sequence for the gap.

pCANTAB5E-c is pCANTAB5E containing the control fragment (Pharmacia Biotech Inc. New Jersey U.S.A).

pAM260 is pCANTAB5E carrying the ScFv cloned from hybridoma 41.11.

Bacterial Strains

The following bacterial strains were used: E. coli HB2151 cells (K12Δ (lac-pro), ara, nal^{r}, thilF′ [proAB, lacI^{q}, lacZΔM15]), (Pharmacia Biotech Inc. New Jersey U.S.A). A Salmonella enteritidis group D isolate was obtained from a clinical sample.

Hybridoma Strains and Growing Conditions

Hybridoma clone 41,11, which expresses a BALB/c monoclonal antibody (IgG1) specific for Salmonella serogroup D, was used [Torensma, R., et al., Appl. Environ, Microbiol. 58:3868(1992)].

The hybridoma cells were grown in IMDM medium, containing 20% fetal calf serum (FCS) and a mixture of penicillin (10 units/ml), streptomycin (10 μg/ml), at 37° C. and 7.5% CO₂.

General Methods in Molecular Biology

Standard molecular biology techniques known in the art and not specifically described were generally followed as in Maniatis et al., Molecular Cloning: A Laboratory Manual, Cold Spring Harbor Laboratory, New York (1989, 1992).

RNA Purification

Hybridoma cells were pelleted and resuspended in TRI-reagent (Telron, Rehovot, Israel). 1 ml TRI-reagent was used for RNA extraction from 5-10×10⁶ cells. After 5 minutes at room temperature, 200 μl of chloroform were added, vigorously mixed, and kept for 5 minute at room temperature, after which centrifugation was performed at 12000×g for 15 minutes at 4° C. The upper phase was transferred to a new tube, 0.5 ml isopropanol was added, mixed, and incubated for 10 minute at room temperature. RNA was precipitated by centrifugation at 12000×g for 8 minute at 4° C. The supernatant was removed, the pellet was washed with 75% ethanol, dried and re-suspended in DEPC treated water.

cDNA Synthesis and Construction of the Plasmid Encoding for the ScFv

A mixture consisting of 5 μl of the RNA preparation (˜10 μg), 0.5 μl oligo dT (100 pmol/μl) and 4.5 μl DEPC water was incubated for 2 minutes at 94° C. and chilled on ice for 2 minutes. Subsequently, 2.5 μl of 10 mM dNTP mix, 1 μl 40 units/μl Rnasin, 5 μl of reverse transcriptase buffer, 10 units/μl of AMV reverse transcriptase (Promega, Medison Wis. U.S.A.) and 5 μl DEPC treated-water were added and the reaction was incubated for 1 hour at 42° C.

The PCR conditions for amplification of fragments were as follows: the reaction mixture consisted of 10 μl cDNA, 1 μl 10 mM dNTP mix, 2 μl of each primer, 0.5 μl 3.5 units/μl High Fidelity Expand (Roche Molecular Biotechnology, Germany), 5 μl buffer and 29.5 μl DEPC-treated water. Amplification conditions were 5 minutes at 94° C., 2 minutes at 50° C., 3 minutes at 74° C., and 28 cycles consisting of 1 minute at 94° C., 2 minutes at 50° C. and 3 minutes at 74° C. The final cycle was: 1 minute at 94° C., 2 minutes at 50° C. and 5 minutes at 74° C.

Growth Conditions and Expression of ScFu by E. coli

HB2151 E. coli bacteria were transformed with pAM260 and grown overnight using 2YT medium that contained 1% glucose and 100 μg/ml ampicillin at 37° C. Subsequently, aliquots of the overnight cultures were grown at 37° C. When cultures reached OD⁶⁰⁰ of 0.8, the bacteria were precipitated by centrifugation for 10 min at 1500×g at room temperature and resuspended in 2YT medium containing 0.4M sucrose, 100 μg ampicillin and 1 mM IPTG. Growth was continued at room temperature for 18-20hr. Following culture the bacteria were centrifuged for 20 min at 1500×g at 4° C. and the supernatant was collected in order to detect the secretion of ScFv. The bacterial pellet was resuspended in PBS containing 1 mM EDTA and stirred for at least 2 hr. at 4° C. The periplasmic extract was collected after centrifugation for 20 min at 10000×g at 4° C.

Western Blotting

Proteins were analysed using SDS-PAGE (12%) and Western blotting. Proteins were transferred by electrophoresis (100 ml MeOH, 0.188 g SDS 2.91 gr Tris 1.47 gr Glycine) to Hybond-C nitro-cellulose membranes (Amersham, Little Chalfont, Enagland). The membranes were then blocked with non-fat milk for 30 min.

ScFv was detected using HEP-conjugated anti-E tag (Pharmacia) that wvas diluted 1:1000 in non-fat milk. For detection of the signal the membrane was stained.

Intact Salmonella ELISA

ELISA was performed using intact fixed Salmonella. For the preparation of the ELISA plates, 10⁹ bacteria/ml were suspended in carbonate buffer pH 9.6 containing NaOH and Na₂CO₃ and added to the plates for 1 hour at 37° C., followed by an overnight incubation at 4° C. Subsequently, plates were extensively washed with PBS and the hybridoma growth medium or bacterial periplasmic fluids were added. The plates were incubated with the fluids for 1 hour at 37° C., and washed with PBS. Either goat anti-mouse (Sigma) or anti-E tag antibodies (Pharmacia) were added and the plates were incubated for an additional 1 hour at 37° C. Subsequently, 1M of the substrate 3,3′5,5′-tetramethylbenzidene (TMB), liquid substrate system (Sigma) was added for 10 minutes at 37° C. The reaction was stopped by the addition of 2N H₂SO₄ and 150 μl and OD readings were quantitated by spectrophotometry.

In Vitro Salmonella Invasion Assay

HT-29 test cells (ATCC Rockville Md.) were grown as confluent monolayers. Salmonella at a concentration of 10⁴/ml, were pretreated for 1 hour at 37° C. with either the growth media of hybridoma 41.11, or with bacterial periplasmic fluids, and added to the cells for an incubation of 24 hrs. Cells were then extensively washed with culture medium containing 50 ng/ml gentamycin and incubated overnight. Following incubation, the supernatant was collected and assayed for IL-8 concentrations. The cells were lysed using distilled H₂O, and the lysate inoculated into Salmonella culture medium for 8 hrs, after which OD readings of the cultures were determined.

In additional inhibitory experiments, bacteria expressing the ScFv, or control plasmids were added to the cells prior to the addition of the Salmonella. Thereafter, the Salmonella were added to the culture and incubated as described herein above.

IL-8 ELISA and Cell Viability Assay

IL-8 was measured by ELISA as previously described [Helm, J. D., et al., (1999) ibid.; Eckmann, L., et al., Gastroenterology 105:1689 (1993)]. Briefly, 96-well plates were coated with polyclonal goat anti-human IL-8 antibodies (R&D Systems, Minneapolis, Minn.), as capturing antibodies. Following incubation with the tested supernatants the cells were washed and polyclonal rabbit anti-human antibodies (Endogen, Boston, Mass.) were added as detecting antibodies. Alkaline phosphatase-labeled mouse anti-rabbit IgG (Sigma) was used as a secondary antibody. The plates were washed and the substrate was added. Results were read using p-nitrophenylphosphate (Sigma).

In vivo Salmonella Colonization Assay

HB2151 bacteria carrying PCANTAB5E-c and HB2151 carrying pAM260 were grown overnight in 2YT medium. Dilutions (1/50) of the cultures were grown to OD⁶⁰⁰ of 0.3 and centrifuged at 3500×rpm for 15 min. The pellet was re-suspended in an induction medium consisting of 2YT supplemented with 0.4M sucrose and 1 mM IPTG and incubated for 2.5 hrs. with shaking at 200×rpm at room temperature.

Each E. coli inoculum consisted of 0.1 ml containing about 1×10⁷ CFU and that of Salmonella about 1×10⁵ CFU. Salmonella was introduced one hour after E. coli was introduced. Bacteria were introduced directly into the digestive system of the chicks using a short plastic tube connected to a syringe.

Experimental and control groups were maintained in separate isolators. Careful measures were taken to prevent cross contamination. Chicks were fed with sterile food and water containing 1 mM IPTG. One day after bacterial inoculation, 8 chicks from each group were sacrificed, cecal samples weighing 0.5 grams were dissected, resuspended in 1 ml sterile PBS and homogenised. 100 μl of the homogenate were inoculated into PBS medium and stored at 4° C. for 30-60 min., until plating. Samples were plated on selective Chromagar media [Gaillot, O., et al., J. Clin. Microbiol. 37.762 (1999)].

### Example 1

Anti-Salmonella ScFv Expressed in E. coli is Secreted to the Periplasm and Growth Medium and Retains Anti-Salmonella Immune Reactivity

Construction of pAM260

In order to create a construct expressing the ScFv for Salmonella, the hybridoma clone 41.11 expressing a BALBc mouse monoclonal antibody (IgG1) specific for Salmonella serogroup D was used [Torensma, R., et al., (1992) ibid.].

RT-PCR was performed in cDNA obtained from these cells, using the primers that are depicted in Table 1. The variable heavy chain was amplified using heavy chain-specific primers (Pharmacia) which were complementary to the 5′ end of the molecule (also denoted by SEQ ID NO: 13) and a second primer that was complementary to the 3′ end of the variable heavy chain (also denoted by SEQ ID NO: 14). For the variable light chain amplification, a primer complementary to the Kappa constant region (also denoted by SEQ ID NO: 9) and a VI-specific, degenerate primer (also denoted by SEQ ID NO: 10) were used.

Next, the amplified fragments were size separated by agarose gel electrophoresis. DNA fragments of the appropriate size were purified from the gel and cloned separately into the pGEMT vector (Promega) to generate pGEMT-VL and pOEMT-VH. The vectors pGEMT-VL and pGEMT-VH were used as a template for an additional PCR amplification using the primers: Sfi I 5′VL (also denoted by SEQ ID NO: 11) and SalI 3′ VL (also denoted by SIQ ID NO: 12) that were specific for the variable light chain and the linker 5′VH, and NotI 3′VH, for the variable heavy chain.

The PCR fragments were digested with the appropriate restriction enzymes (SfiI, SalI, NotI) and purified using microspin columns (S400, Pharmacia), after which they were ligated. The resulting ScFv fragment was amplified by PCR with the primers SfiI 5′VL and NotI 3′VH and ligated into the pCANTAB5E vector. The final vector containing the ScFv was termed pAM260.

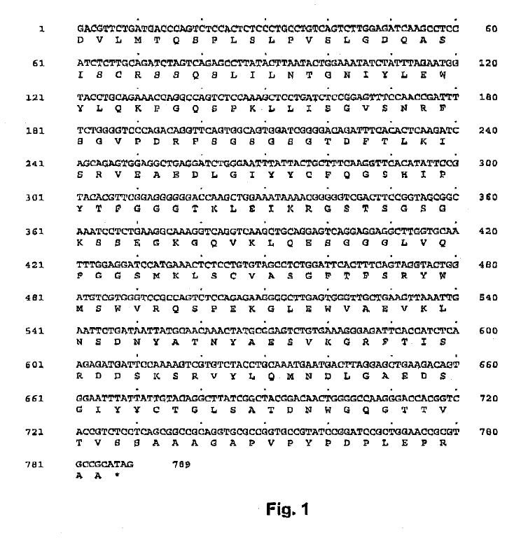

Shown in FIG. 1 are the nucleotide sequences corresponding to the different fragments that were used for the construction of the ScFv. cDNAs encoding the variable light and variable heavy chain domains were linked with a short glycine/serine polypeptide and cloned between the Lac-z promoter and the TAB epitope tag in the pCANTAB5E plasmid, to create pAM260.

TABLE 1
List of primers used in the PCR for the construction of pAM260

| | |
|---|---|
| kappa constant | 5′ GCG CCG TCT AGA ATT AAC ACT CAT TCC TGT TGA A 3′ |
| SEQ ID NO: 9 | |
| VL-degenerate SEQ ID NO: 10 | 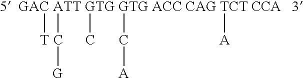 |
| Sfi I 5′vl SEQ ID NO: 11 | 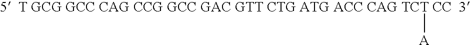 |
| Sal I 3′vl | 5′ CG GGT CGA CCC CCG TTT TAT TTC CAG CTT GGT CCC 3′ |
| SEQ ID NO: 12 | |
| linker 5′vh SEQ ID NO: 13 | 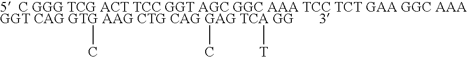 |
| Not I 3′vh | 5′ ATT TGC GGC CGC TGA GGA GAC GGT GAC CGT GG 3′ |
| SEQ ID NO: 14 | |

Expression of pAM260

To express the construct of the invention the plasmid was introduced into the nonsuppressor bacteria strain HB2151, in which the stop codon is recognized and protein synthesis is aborted at the end of the ScFv gene, so that the g3p fusion protein is not produced. The resulting ScFv protein was transformed into the periplasmic space, but was not assembled into a phage particle, since it lacked the gene 3 domain. Instead, a soluble antibody fragment accumulated in the periplasm and leaked into the medium.

Antibody fragments may aggregate in bacterial cells due to high level of expression [Bowden, G. A. et al., J. Biol. Chem. 265:16760 (1990)].

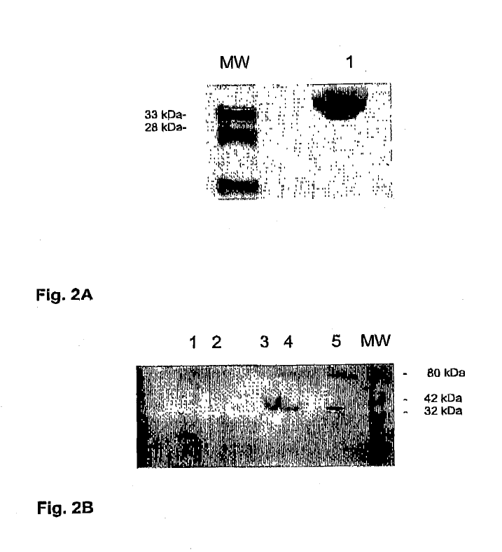

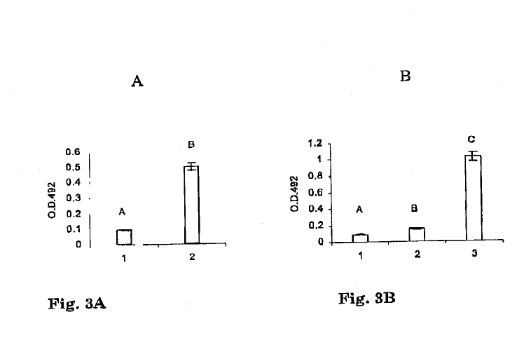

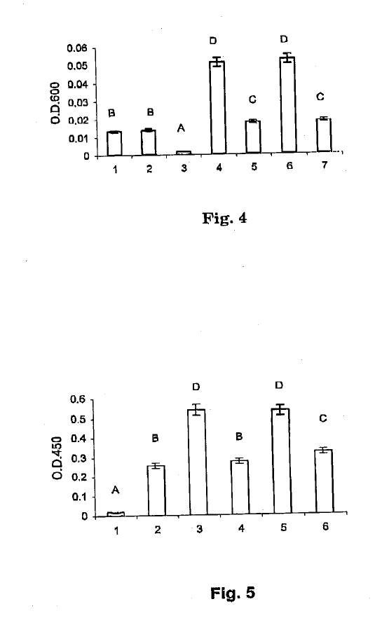

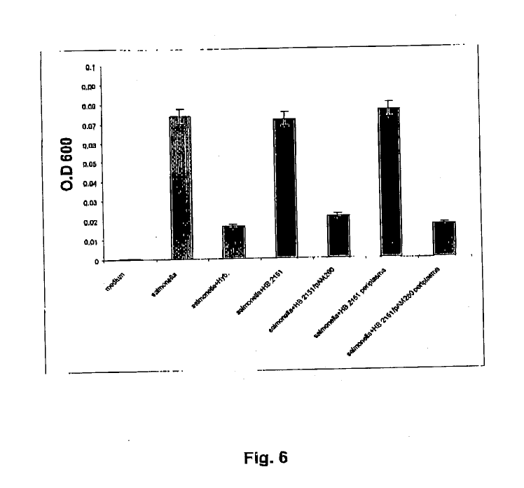

Growing the bacteria in the presence of raffinose or sucrose [Bowden, G. A. et al,, (1990) !bid.; Kipriyanov, S. M., et al., J. Immunol. Methods 200:69 (1997)] can reduce such aggregation of antibody fragments in E. coli. Therefore, induction of ScFv secretion from E. coli HB2151 containing pAM260 was carried out in the presence of sucrose. As shown in FIG. 2, Western blot analysis detected a 33 kd protein which was recognized by the anti-TAB epitope tag antibody and corresponded to the ScFv. This protein was detected both in the bacterial periplasm (2A) and in the culture medium (2B). Of note is that induction of ScFv secretion by sucrose was not significantly better than with glucose. However, the experiments were performed with sucrose induction. Culture media of bacteria that were transformed with the control vector and grown in the presence of sucrose or glucose served as negative controls.

Recombinant molecules that are expressed within bacteria may lose their original biologic function. To test whether the ScFv retained its affinity for Salmonella serogroup D bacteria, periplasmic fluids obtained from E. coli that expressed recombinant ScFv were tested by ELISA for their ability to detect whole Salmonella. Concentrated growth media of hybridoma 41.11 served as a positive control. As shown in FIG. 3, the ScFv detected Salmonella that were fixed to the ELISA plates. These results indicated that the recombinant antibody retained its antigenic specificity. Of note, since the anti-Salmonella hybridoma antibody and the ScFv were not purified, the exact quantity and affinity of the antibodies could not be determined and the results are presented as OD measurements.

### Example 2

ScPv Antibodies Reduce Invasion of Salmonella into HT-29 Cells and Post-Invasion IL-8 Secretion

Invasion Experiments

Salmonella penetrate into intestinal epithelial cells in culture. Thus, the anti-Salmonella ScFv were tested for their ability to prevent the invasion of Salmonella into monolayers of HT-29 human intestinal epithelial cells. FIG. 4 shows that pre-treatment of Salmonella with ScFv reduced the number of bacteria that could be grown from the cell lysate in about one order of magnitude compared to cells that were infected with bacteria and were not treated with the ScFv. The results were comparable to those obtained following the treatment of the bacteria with the native hybridoma antibody. No protective effect was noted in cells that were incubated with the periplasmic extract of the control bacteria that did not express ScFv, or in cells that were incubated with control hybridoma growth medium. These results suggest that the antibody was efficient in preventing the entry of the bacteria into the cells.

IL-8 Secretion

Previous studies have shown that the invasion of Salmonella into intestinal epithelial cells resulted in secretion of pro-inflammatory cytokines and chemokines by the cells [Eckmann, L, et al., Gastroenterology 105:1689 (1993); Eckmann, L., et al., J. Clin. Invest. 100:296 (1997)]. Therefore, the concentration of the chemokine IL-8 was determined in the culture media of HT-29 cells that were pre-treated with antibodies and controls following exposure to Salmonella. As shown in FIG. 5, compared to control periplasmic fluid, the periplasmic fluid containing ScFv significantly reduced the amount of IL-8 that was secreted from the HT-29 cells. These results further support the biologically significant protective effect of ScFv against Salmonella invasion.

Further, the results obtained using an in vitro model system demonstrated that the ScFv antibody significantly protected the epithelial cells from bacterial invasion. In addition, following co-incubation with Salmonella, the antibody also reduced secretion of the pro-inflammatory chemokine IL-8. Such effect would likely ameliorate tissue damage that can potentially occur by an inflammatory response induced by bacterial invasion. Furthermore, the beneficial effects using a cell line indicate that the ScFv was highly protective even in the absence of a fully functioning mucosal immune system.

Bacterial Expressed ScFv Reduce Invasion of Salmonella into HT29 Cells

The feasibility of reducing the invasion of Salmonella using bacterial expressed ScFv, was next examined. Following culture, E. coli were induced by IPTG to express the ScFv, whereas E. coli containing the vector only served as negative controls, Following IPTG induction, 4.8×10⁸ E. coli were co-cultured with 4×10⁵ Rifampicin-sensitive Salmonella for 1 hour at 37C.° Bacterial culture was then added for 2 hours at 37 C.° to confluent HT-29 cells. Supernatants from hybridoma 41.11 and periplasmic fluid from bacteria expressing the ScFv served as positive controls. Periplasmic fluid from control E. coli served an additional negative control. Subsequently, the cells were extensively washed with medium containing rifampicin, after which the culture was continued for 3 hours. Thereafter, the cells were lysed using hypotonic shock and intracellular Salmonella were inoculated for culture in brain heart liquid medium. Bacteria were quantitated by OD₆₀₀ measurements. As shown in FIG. 6, bacteria-expressed SvFc antibodies significantly reduced invasion of Salmonella to HT29 cells, similarly to the hybridoma or the periplasmic fluid from bacteria expressing the ScFv. Negative controls showed no reduction in Salmonella invasion. Thus, the ScFv of the invention inhibits invasion of Salmonella, when expressed in bacteria.

### Example 3

ScFv Antibodies Reduce Intestinal Colonization of Chickens by Salmonella

One-day old chickens are easily colonised by Salmonella [Nurmi, E. et al., Nature 241:210 (1973)]. Thus, an optimal imnmunisation should be efficient in significantly reducing bacterial load in the chicks and prevent tissue invasion.

Therefore, the ability of live bacteria that continuously express the anti-Salmonella ScFv to protect one day old-chicks from colonization of the intestinal tract with Salmonella and to prevent the invasion of intestinal tissue and the resulting liver abscesses was tested. One day old, specific pathogen free (SPF) chickens were divided into the following experimental groups:

Group A—was challenged with Salmonella one hour after the experiment had begun.

Group B—was challenged with E. coli carrying pAM260 at the beginning of the experiment.

Group C—was challenged with E. coli carrying pCANTAB5E-c at the begining of the experiment. One hour later the chicks were challenged with Salmonella.

Group D—was challenged with E. coli carrying pAM260 at the beginning of the experiment, and an hour later challenged with Salmonella.

The chicks were fed with 10⁷ CFU of E. coli that expressed the ScFv and were challenged with 10⁵ Salmonella one hour later. Prior to inoculation into the chicks, the E. coli bacteria were induced to produce ScFv by addition of IPTG to the growth media. The drinking water was supplemented also with IPTG, in order to continuously induce the production of ScFv even after the bacteria colonized the intestinal tract of the chicks. As shown in Table 2, administration of E. coli that contained pAM260 significantly reduced the number of Salmonella within the cecal homogenates. The number of Salmonella was reduced by nearly one fold compared to the number of Salmonella that were found in intestinal tissue of chicks that received plasmid-free E. coli. These findings demonstrate that the effect of the ScFv was significantly protective also in vivo.

TABLE 2
Salmoneila survival in chick's cecal tissue
A - Salmonella colonies

| CHICKEN | | | | | | | | |
|---|---|---|---|---|---|---|---|---|
| GROUP | 1 | 2 | 3 | 4 | 6 | 6 | 7 | 8 |
| A | >300 | 276 | 301 | 294 | 260 | 320 | 331 | N.S. |
| B | N.S. | 185 | N.S. | N.S. | N.S. | N.S. | N.S. | N.S. |
| C* | >300 | 212 | >300 | 226 | 284 | 216 | >300 | >300 |
| D* | 6 | 27 | 23 | 21 | 18 | 26 | 23 | 27 |

*Statistically significant different p < 0.05 (p = 3.52E−07)
>300 was calculated as = 300

B - E. coli colonies

| CHICKEN | | | | | | | | |
|---|---|---|---|---|---|---|---|---|
| GROUP | 1 | 2 | 3 | 4 | 5 | 6 | 7 | 8 |
| A | N.E. | N.E. | N.E. | N.E. | N.E. | N.E. | N.E. | N.E. |
| B | >300 | >300 | >800 | >300 | >300 | >300 | >300 | >300 |
| C | >300 | >300 | >300 | >300 | >300 | >300 | >300 | >300 |
| D | >300 | >300 | >300 | >300 | >300 | N.E. | >300 | >800 |

N.S = no Salmonella
N.E = no E. coli

The results presented herein demonstrated that bacteria, which resided within the intestine and were engineered to express ScFv, were able to provide protection against intestinal pathogens.

Inoculation of the intestinal tract of one day old-chickens with E. coli that expressed anti-Salmonella ScFv, significantly reduced the colonisation of Salmonella within the intestine and provided protection from mucosal invasion and liver infection. This effect was clear, even though, due to methodological considerations, a high number of Salmonella was used for the inoculum. Predictably, if the chickens were exposed to smaller numbers of Salmonella similar to natural conditions, the protective effect would be even better.

1. A composition for conferring to a subject immunity against a gastrointestinal (GI) pathogen comprising as an active ingredient at least one microorganism transformed with at least one vector comprising a nucleic acid sequence coding for an immunoglobulin-like polypeptide having specific binding affinity for said pathogen and optionally comprising pharmaceutically or veterinarily acceptable carrier, adjuvant or diluent.

2. The composition of claim 1, wherein said pathogen is any one of human and animal gastrointestinal enterobacterium.

3. The composition of claim 2, wherein said gastrointestinal enterobacterium is a Salmonella species.

4. The composition of claim 3, wherein said Salmonella is Salmonella enteriditis serogroup D.

5. The composition of any one of claims 1 to 4, wherein said immunoglobulin-like polypeptide is a single chain variable fragment (ScFv) antibody having binding affinity to gastrointestinal enterobacteria, said single chain variable fragment antibody (ScFv) consisting essentially of:
a. a first polypeptide comprising the binding portion of the light chain variable region of an antibody;
b. a second polypeptide comprising the binding portion of the heavy chain variable region of an antibody; and
c. a polypeptide linker linking said first and second polypeptides (a) and (b).

6. The composition of claim 5, wherein said single chain variable fragment antibody (ScFv) is encoded by the nucleic acid sequence substantially as denoted by SEQ ID NO:1, and consisting essentially of:
a. a first polypeptide comprising the binding portion of the light chain variable region of an antibody against Salmonella enteriditis serogroup D, said polypeptide being encoded by the nucleic acid sequence substantially as denoted by SEQ ID NO:2;
b. a second polypeptide comprising the binding portion of the heavy chain variable region of an antibody against Salmonella enteriditis serogroup D, said polypeptide being encoded by the nucleic acid sequence substantially as denoted by SEQ ID NO:3; and
c. a glycine/serine polypeptide linker linking said first and second polypeptides, encoded by the nucleic acid sequence substantially as denoted by SEQ ID NO:4.

7. The composition of claim 6, wherein said transformed microorganism is any one of prokaryotic and eukaryotic microorganism.

8. The composition of claim 7, wherein said microorganism is any one of bacteria and yeast.

9. The composition of claim 8, wherein said bacterium is E. coli.

10. The composition of claim 1, wherein said subject is any one of human and domestic animal.

11. The composition of claim 10, wherein said domestic animal is a domestic bird.

12. An isolated DNA molecule coding for an immunoglobulin-like polypeptide having specific binding affinity for a gastrointestinal (GI) pathogen.

13. The DNA of claim 12, wherein said pathogen is a human or animal gastrointestinal (GI) enterobacterium.

14. The DNA of claim 13, wherein said immunoglobulin-like polypeptide is a single-chain variable fragment (ScFv) antibody.

15. The DNA according to claim 14, wherein said single-chain variable fragment antibody has specific affinity for a gastrointestinal pathogen, said single-chain variable fragment antibody consisting essentially of:
a. a first polypeptide comprising the binding portion of the light chain variable region of an antibody;
b. a second polypeptide comprising the binding portion of the heavy chain variable region of an antibody; and
c. a polypeptide linker linking said first and second polypeptides (a) and (b).

16. The DNA of claim 15, wherein said gastrointestinal pathogen is Salmonella species.

17. The DNA of claim 16, wherein said gastrointestinal pathogen is Salmonella enteriditis serotype D.

18. The DNA of claim 17, being the DNA substantially as denoted by SEQ ID NO:1.

19. A replicable cloning or expression vehicle comprising the DNA molecule of any one of claims 12 to 18.

20. The vehicle of claim 19, which is the pAM20 phagemid substantially as specified in the Examples.

21. A host cell transformed with the vehicle of any one of claims 19 and 20.

22. The host cell of claim 21, which is any one of a prokaryotic and eukaryotic cell.

23. The host cell of claim 22, which is any one of bacterial and yeast cell.

24. The host cell of claim 23, wherein said bacterium is E. coli.

25. The host cell of any one of claims 21 to 24, capable of in vivo producing a biologically active immunoglobulin-like polypeptide that recognizes and specifically binds to a gastrointestinal enteropathogen.

26. The host cell of claim 25, wherein said immunoglobulin-like polypeptide is a single-chain variable fragment (ScFv) antibody that recognizes and specifically binds to said gastrointestinal pathogen.

27. The host cell of claim 26, capable of in vivo producing a single-chain variable fragment (ScFv) antibody that blocks colonization of gastrointestinal pathogens in the intestinal tract of an animal, when introduced to said animal.

28. A single-chain variable fragment (ScFv) antibody molecule having affinity for Salmonella species, said single-chain variable fragment antibody consisting essentially of:
a. a first polypeptide comprising the binding portion of the light chain variable region of an antibody;
b. a second polypeptide comprising the binding portion of the heavy chain variable region of an antibody; and
c. a polypeptide linker linking said first and second polypeptides (a) and (b).

29. The single-chain variable fragment antibody molecule of claim 28, encoded by the nucleic acid sequence substantially as denoted by SEQ ID NO:1.

30. A method of treating infections of gastrointestinal enteropathogens in a subject in need of such treatment, comprising the administration to said subject of microorganisms transformed with DNA encoding at least one immunoglobulin-like polypeptide or with an expression vector comprising the same or of pharmaceutically or veterinary composition comprising the same, wherein said polypeptide has specific binding affinity to said pathogen.

31. The method of claim 30, for treating infections of gastrointestinal enteropathogenic bacteria in a subject, comprising the administration to said subject of microorganisms transformed with DNA encoding at least one single-chain antibody or of an expression a vector comprising the same or of a pharmaceutical or veterinary composition comprising the same, said single-chain antibody comprises:
a. a first polypeptide comprising the binding portion of the light chain variable region of an antibody;
b. a second polypeptide comprising the binding portion of the heavy chain variable region of an antibody; and
c. a polypeptide linker linking said first and second polypeptides (a) and (b).

32. The method of any one of claims 30 and 31, wherein said subject is any one of human or domestic animal.

33. The method of claim 32, wherein said gastrointestinal enterobacterium is Salmonella species.

34. The method of claim 33, wherein said single-chain variable fragment (ScFv) antibody is encoded by the nucleic acid sequence substantially as denoted by SEQ ID NO:1, and consists essentially of:
a. a first polypeptide comprising the binding portion of the light chain variable region of an antibody against Salmonella enteriditis serogroup D, said polypeptide being encoded by the nucleic acid sequence substantially as denoted by SEQ ID NO:2;
b. a second polypeptide comprising the binding portion of the heavy chain variable region of an antibody against Salmonella enteriditis serogroup D, said polypeptide being encoded by the nucleic acid sequence substantially as denoted by SEQ ID NO:3; and
c. a glycine/serine polypeptide liner linking said first and second polypeptides (a) and (b) encoded by the nucleic acid sequence substantially as denoted by SEQ ID NO:4.

35. The method of claim 30, wherein upon administration to said subject said microorganism in vivo produces an immunoglobulin-like polypeptide in said subject, which polypeptide recognizes and specifically binds to an enterogenic pathogen.

36. The method of claim 35, wherein upon administration to said subject said microorganism in vivo produces a single-chain variable fragment antibody in said subject intestine, which antibody recognizes and specifically binds to a gastrointestinal pathogen.

37. The method of claim 36, wherein said single-chain variable fragment antibody blocks colonization of gastrointestinal pathogens in the intestinal tract of said subject.

38. The method of claim 37, wherein said subject is any one of human or domestic animal.

39. The method of claim 38, wherein said domestic animal is a domestic bird.

40. The method of any one of claims 30 to 39, wherein said microorganism is any one of prokaryotic and eukaryotic microorganisms.

41. The method of claim 40, wherein said microorganism is any one of bacteria and yeast.

42. The method of claim 41, wherein said bacteria is E. coli.

43. The method of any one of claims 30 to 42, wherein said composition is administered via drinking water, feed, spraying, oral gavage and/or directly into the digestive tract.

44. The method of any one of claims 30 to 43, further comprising the step of controlling the expression of said single-chain variable fragment antibody by continuously inducing the production of said single-chain variable fragment antibody.


SEQUENCE LISTING (14 sequences)

SEQ ID NO: 1 - DNA, 789 bp, Mus musculus
gacgttctgatgacccagtctccactctccctgcctgtcagtcttggagatcaagcctccatctcttgcagatctagtcagagccttatacttaatactggaaatatctatttagaatggtacctgcagaaaccaggccagtctccaaagctcctgatctccggagtttccaaccgattttctggggtcccagacaggttcagtggcagtggatcggggacagatttcacactcaagatcagcagagtggaggctgaggatctgggaatttattactgctttcaaggttcacatattccgtacacgttcggaggggggaccaagctggaaataaaacgggggtcgacttccggtagcggcaaatcctctgaaggcaaaggtcaggtcaagctgcaggagtcaggaggaggcttggtgcaatttggaggatccatgaaactctcctgtgtagcctctggattcactttcagtaggtactggatgtcgtgggtccgccagtctccagagaaggggcttgagtgggttgctgaagttaaattgaattctgataattatgcaacaaactatgcggagtctgtgaaagggagattcaccatctcaagagatgattccaaaagtcgtgtctacctgcaaatgaatgacttaggagctgaagacagtggaatttattattgtacaggcttatcggctacggacaactggggccaagggaccacggtcaccgtctcctcagcggccgcaggtgcgccggtgccgtatccggatccgctggaaccgcgtgccgcatag

SEQ ID NO: 2 - DNA, 339 bp, Mus musculus
gacgttctgatgacccagtctccactctccctgcctgtcagtcttggagatcaagcctccatctcttgcagatctagtcagagccttatacttaatactggaaatatctatttagaatggtacctgcagaaaccaggccagtctccaaagctcctgatctccggagtttccaaccgattttctggggtcccagacaggttcagtggcagtggatcggggacagatttcacactcaagatcagcagagtggaggctgaggatctgggaatttattactgctttcaaggttcacatattccgtacacgttcggaggggggaccaagctggaaataaaacgg

SEQ ID NO: 3 - DNA, 351 bp, Mus musculus
caggtcaagctgcaggagtcaggaggaggcttggtgcaatttggaggatccatgaaactctcctgtgtagcctctggattcactttcagtaggtactggatgtcgtgggtccgccagtctccagagaaggggcttgagtgggttgctgaagttaaattgaattctgataattatgcaacaaactatgcggagtctgtgaaagggagattcaccatctcaagagatgattccaaaagtcgtgtctacctgcaaatgaatgacttaggagctgaagacagtggaatttattattgtacaggcttatcggctacggacaactggggccaagggaccacggtcaccgtctcctca

SEQ ID NO: 4 - DNA, 41 bp, Artificial Sequence
  Description of Artificial SequenceDNA sequence coding for the polypeptide linker
gggtcgacttccggtagcggcaaatcctctgaaggcaaagg

SEQ ID NO: 5 - protein, 262 aa, Mus musculus
DVLMTQSPLSLPVSLGDQASISCRSSQSLILNTGNIYLEWYLQKPGQSPKLLISGVSNRFSGVPDRFSGSGSGTDFTLKISRVEAEDLGIYYCFQGSHIPYTFGGGTKLEIKRGSTSGSGKSSEGKGQVKLQESGGGLVQFGGSMKLSCVASGFTFSRYWMSWVRQSPEKGLEWVAEVKLNSDNYATNYAESVKGRFTISRDDSKSRVYLQMNDLGAEDSGIYYCTGLSATDNWGQGTTVTVSSAAAGAPVPYPDPLEPRAA

SEQ ID NO: 6 - protein, 113 aa, Mus musculus
DVLMTQSPLSLPVSLGDQASISCRSSQSLILNTGNIYLEWYLQKPGQSPKLLISGVSNRFSGVPDRFSGSGSGTDFTLKISRVEAEDLGIYYCFQGSHIPYTFGGGTKLEIKR

SEQ ID NO: 7 - protein, 117 aa, Mus musculus
QVKLQESGGGLVQFGGSMKLSCVASGFTFSRYWMSWVRQSPEKGLEWVAEVKLNSDNYATNYAESVKGRFTISRDDSKSRVYLQMNDLGAEDSGIYYCTGLSATDNWGQGTTVTVSS

SEQ ID NO: 8 - protein, 14 aa, Artificial Sequence
  Description of Artificial SequencePolypeptide linker
GSTSGSGKSSEGKG

SEQ ID NO: 9 - DNA, 34 bp, Artificial Sequence
  Description of Artificial SequencePrimer for kappa constant
gcgccgtctagaattaacactcattcctgttgaa

SEQ ID NO: 10 - DNA, 24 bp, Artificial Sequence
  Description of Artificial SequencePrimer for VL-degenerate , Y in position 3 may be C or T, V in positions 4 and 10 may be A or C or G,  W  in position  19 may be A or T
gayvttgtgvtgacccagwctcca

SEQ ID NO: 11 - DNA, 39 bp, Artificial Sequence
  Description of Artificial SequenceSfi I 5′ VL Primer. w in position 37 may be A or  T.
tgcggcccagccggccgacgttctgatgacccagtcwcc

SEQ ID NO: 12 - DNA, 35 bp, Artificial Sequence
  Description of Artificial SequenceSal I 3′ VL Primer
cgggtcgacccccgttttatttccagcttggtccc

SEQ ID NO: 13 - DNA, 66 bp, Artificial Sequence
  Description of Artificial Sequencelinker 5′ VH Primer, S  in position 49 may be G or  C, S  in position 59 may be G or C and w  in position 64 may be A or T.
cgggtcgacttccggtagcggcaaatcctctgaaggcaaaggtcaggtsaagctgcagsagtcwgg

SEQ ID NO: 14 - DNA, 32 bp, Artificial Sequence
  Description of Artificial SequenceNot I 3′ VH Primer
atttgcggccgctgaggagacggtgaccgtgg